(19) United States
(12) Patent Application Publication
(10) Pub. No.: US 2022/0391753 A1
(43) Pub. Date: Dec. 8, 2022

(54) METHOD FOR DETERMINING TRAINING STATUS SELECTED FROM A SET OF TRAINING STATUS ALTERNATIVES

(71) Applicant: bOMDIC, Inc., New Taipei (TW)

(72) Inventors: JR-FANG LIOU, New Taipei (TW); HAN-TSUNG PAN, New Taipei (TW)

(21) Appl. No.: 17/342,517

(22) Filed: Jun. 8, 2021

(51) Int. Cl. G06N 20/00

(52) U.S. Cl. CPC G06N 20/00

(57) ABSTRACT

The present invention discloses a method for determining a training status selected from a set of training status alternatives at a current time. Determine an overall training load in a duration before the current time. Determine a first term representing a positive effect on a training performance based on the overall training load and a second term representing a negative effect on the training performance based on the overall training load. Determine a third term representing the training performance based on the first term and the second term. Generate at least one feature based on at least one of the first term, the second term and the third term. Determine one of the set of training status alternatives based on the at least one feature by a classifying model describing the set of training status alternatives are associated with the at least one features.

### BACKGROUND OF THE INVENTION

### 1. Field of The Invention

The present invention relates to a method for train-monitoring, and more particularly to a method for determining a training status selected from a set of training status alternatives.

### 2. Description of Related Art

Determining a training status is very important in the exercise-training. For example, a person needs to know the training status before the competition and he hopes that the training status before the competition is the most optimized for the competition (e.g., peaking). So, the method for monitoring the most optimized training status is needed. The training status is highly associated with the training performance in the exercise procedure. The known method uses a plurality of parameters associated with VO₂ₘₐₓ (the maximum rate of the oxygen consumption) and the training load to determine the training status. However, VO₂ₘₐₓ is one of the indexes evaluating the training performance and it doesn't completely represent the training performance. In other words, the known method can't precisely to determine the training status.

Accordingly, the present invention proposes a method for determining a training status selected from a set of training status to overcome the above-mentioned disadvantages.

### SUMMARY OF THE INVENTION

In other to overcome the problem that VO₂ₘₐₓ doesn't completely represent performance in the known method, the present invention builds up a training performance model to describe the training performance in detail. The training performance is determined in the training performance model based on a positive-effect term for the training performance and a negative-effect term for the training performance defined in the training performance model. The positive-effect term and the negative-effect term are both determined based on the overall training load in a duration before the current time. The present invention further uses a classifying model to determine one of a set of training status alternatives based on at least one feature generated based on at least one of the positive-effect term, the negative-effect term and the training performance. Because the training performance model takes into account a positive effect on the training performance in the positive-effect term and a negative effect on the training performance in the negative-effect term at the same time, a combination of the positive-effect term and the negative-effect term can completely/precisely estimate the training performance. Precisely estimating the training performance further contributes to precisely estimating the training status. Each of the positive-effect term and the negative-effect term has an overall training load setting associates the over training load with corresponding one of the positive-effect term and the negative-effect term. At least one (or both) of the overall training load setting of the first term and the overall training load setting of the second term may be adjusted according to the training performance to precisely estimate the training performance. Precisely estimating the training performance further contributes to precisely estimating the training status.

In one embodiment, the present invention discloses a method for determining a training status selected from a set of training status alternatives each of which has a corresponding physical condition at a current time. The method comprises: determining an overall training load in a duration before the current time based on an exercise intensity measured by a sensing unit; determining, by a processing unit, a first term representing a positive effect on a training performance based on the overall training load and a second term representing a negative effect on the training performance based on the overall training load; determining, by the processing unit, a third term representing the training performance based on the first term and the second term; generating, by the processing unit, at least one feature based on at least one of the first term, the second term and the third term; and determining, by the processing unit, one of the set of training status alternatives based on the at least one feature by a classifying model describing that the set of training status alternatives are associated with the at least one features.

In one embodiment, the present invention discloses an apparatus for determining a training status selected from a set of training status alternatives each of which has a corresponding physical condition at a current time. The apparatus comprises: a processing unit; and a memory unit including a computer program code which, when executed by the apparatus, causes the apparatus to perform a process comprising steps of: determining an overall training load in a duration before the current time based on an exercise intensity measured by a sensing unit; determining a first term representing a positive effect on a training performance based on the overall training load and a second term representing a negative effect on the training performance based on the overall training load; determining, by the processing unit, a third term representing the training performance based on the first term and the second term; generating, by the processing unit, at least one feature based on at least one of the first term, the second term and the third term; and determining, by the processing unit, one of the set of training status alternatives based on the at least one feature by a classifying model describing that the set of training status alternatives are associated with the at least one features.

In one embodiment, the present invention discloses a method for determining a training performance at a current time. The method comprises: determining an overall training load in a duration before the current time based on an exercise intensity measured by a sensing unit; determining, by a processing unit, a first term representing a positive effect on the training performance based on the overall training load and a second term representing a negative effect on the training performance based on the overall training load; and determining, by the processing unit, a third term representing the training performance based on the first term and the second term; wherein the duration is divided into a plurality of time segments each of which comprises a training load therein, wherein for each first time segment of the plurality of time segments, a positive-effect training load is generated for the first term and a negative-effect training load is generated for the second term, wherein the positive-effect training load is determined based on a combination of the training load and a positive-effect weighting factor and the negative-effect training load is determined based on a combination of the training load and a negative-effect weighting factor; wherein the positive-effect weight factor comprises a first positive-effect factor decreasing with an increase of a time interval between the first time segment between the current time and the negative-effect weight factor comprises a first negative-effect factor decreasing with the increase of the time interval between the first time segment between the current time; wherein the positive-effect weight factor further comprises a second positive-effect factor combined with the first positive-effect factor and the negative-effect weight factor further comprises a second negative-effect factor combined with the first negative-effect factor, wherein at least one second time segment is between the first time segment and the current time, wherein the second positive-effect factor takes into account the training load in each of the at least one second time segment being not less than a first threshold or not more than a second threshold and the second negative-effect factor takes into account the training load in each of the at least one second time segment being not less than a third threshold or not more than a fourth threshold, wherein the first threshold is more than the second threshold and the third threshold is more than the fourth threshold.

The detailed technology and above preferred embodiments implemented for the present invention are described in the following paragraphs accompanying the appended drawings for people skilled in the art to well appreciate the features of the claimed invention.

### BRIEF DESCRIPTION OF THE DRAWINGS

The foregoing aspects and many of the accompanying advantages of this invention will become more readily appreciated as the same becomes better understood by reference to the following detailed description when taken in conjunction with the accompanying drawings, wherein:

FIG. 1 illustrates a schematic block diagram of an exemplary apparatus in the present invention;

FIG. 2 illustrates a method for determining a training status selected from a set of training status alternatives at a current time;

FIGS. 3A to 3D illustrate that for each first time segment (each of TS₁, TS₂, TS₃, TS₄, TS₅ and TS₆) of time segments, a positive-effect training load (the corresponding one of PL₁, PL₂, PL₃, PL₄, PL₅ and PL₆) is generated for the first term, a negative-effect training load (the corresponding one of NL₁, NL₂, NL₃, NL₄, NL₅ and NL₆) is generated for the second term, and the positive-effect training load and the negative-effect training load are combined for the third term;

FIGS. 4A to 4C illustrate some cases in which each of the third positive-effect factor and the third negative-effect factor is needed to be taken into account;

FIG. 5 illustrates the original training load is modified to be the training load (e.g., according to the finite training capacity of the body if the original training load is more than a threshold);

FIGS. 6A to 6D illustrate that the training performance model with the different time segment number in the first term and the second term; and

FIG. 7 illustrates some of the training status alternatives and the corresponding the limitations associated with a first trend of the first term, a second trend of the second term and a third trend of the third term.

### DETAILED DESCRIPTION OF THE ILLUSTRATED EMBODIMENTS

The detailed explanation of the present invention is described as following. The described preferred embodiments are presented for purposes of illustrations and description and they are not intended to limit the scope of the present invention.

### Definition of the Terms

Exercise Intensity

The exercise intensity may refer to how much energy is expended when exercising. The exercise intensity may define how hard the body has to work to overcome a task/exercise. Exercise intensity may be measured in the form of the internal workload. The parameter of the exercise intensity associated with the internal workload may be associated with a heart rate, an oxygen consumption, a pulse, a respiration rate and RPE (rating perceived exertion). The exercise intensity may be measured in the form of the external workload. The parameter of the exercise intensity associated with the external workload may be associated with a speed, a power, a force, a motion intensity, a motion cadence or other kinetic data created by the external workload resulting in energy expenditure. The heart rate may be often used as a parameter of the exercise intensity.

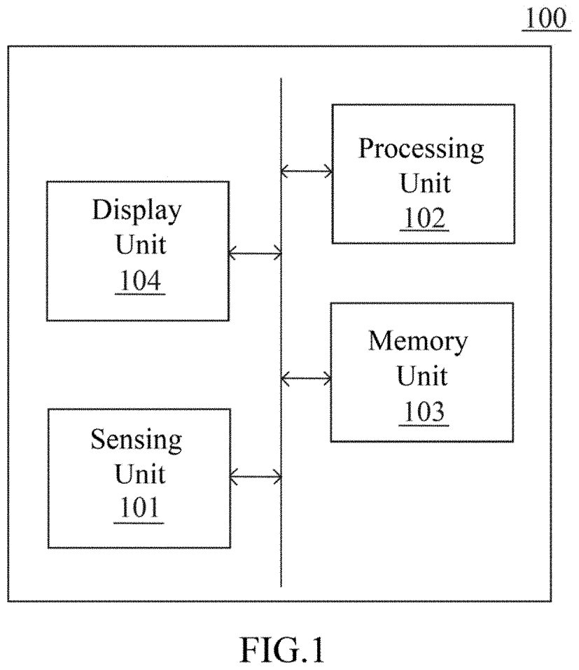

The method in the present invention may be applied in all kinds of apparatuses, such as a measurement system, the device worn on the individual (e.g., the device attached to the wrist belt or chest belt), a wrist top device, a mobile device, a portable device, a personal computer, a server or a combination thereof. FIG. 1 illustrates a schematic block diagram of an exemplary apparatus 100 in the present invention. The apparatus 100 may comprise a sensing unit 101 (e.g., at least one sensor), a processing unit 102, a memory unit 103 and a displaying unit 104. One unit may communicate with another unit in a wired or wireless way. The apparatus 100 may comprise at least one device; the sensing unit 101 may be in one device (e.g., the device worn on the individual or watch) and the processing unit 102 may be in another device (e.g., mobile device or mobile phone); the sensing unit 101 and the processing unit 102 may be in a single device (e.g., the device worn on the individual or watch). The sensing unit 101 may be attached to/comprised in a belt worn on the individual. The sensing unit 101 may be a sensor (e.g., heart activity sensor) which may measure a signal associated with the physiological data, the cardiovascular data or the internal workload from the person's body. The signal may be measured by applying a skin contact from chest, wrist or any other human part. The sensing unit 101 may comprise a second sensor (e.g., motion sensor) which may measure the exercise intensity associated with the external workload. The second sensor may comprise at least one of an accelerometer, a magnetometer and a gyroscope. The sensing unit 101 may further comprise a position sensor (e.g., GPS: Global Positioning System). The sensing unit 101 may comprises at least two sensors described above. The processing unit 102 may be any suitable processing device for executing software instructions, such as a central processing unit (CPU). The processing unit 102 may be a computing unit. The apparatus 100 may comprise at least one device; a first portion of the computing unit may be in one device (e.g., the device worn on the individual or watch), a second portion of the computing unit may be in another device (e.g., mobile device or mobile phone) and a first portion of the computing unit may communicate with a second portion of the computing unit in a wired or wireless way; a first portion of the computing unit and a second portion of the computing unit may be in a single device (e.g., the device worn on the individual or watch). The memory unit 103 may include random access memory (RAM) and read only memory (ROM), but it is not limited to this case. The memory unit 103 may include any suitable non-transitory computer readable medium, such as ROM, CD-ROM, DVD-ROM and so on. Also, the non-transitory computer readable medium is a tangible medium. The non-transitory computer readable medium includes a computer program code which, when executed by the processing unit 102, causes the apparatus 100 to perform desired operations (e.g., operations listed in claims). The display unit 104 may be a display for displaying the training performance and the training status at the current time. The displaying mode may be in the form of words, a voice or an image. The sensing unit 101, the processing unit 102, the memory unit 103 and the displaying unit 104 in the apparatus 100 may have any suitable configuration and it doesn't be described in detail therein.

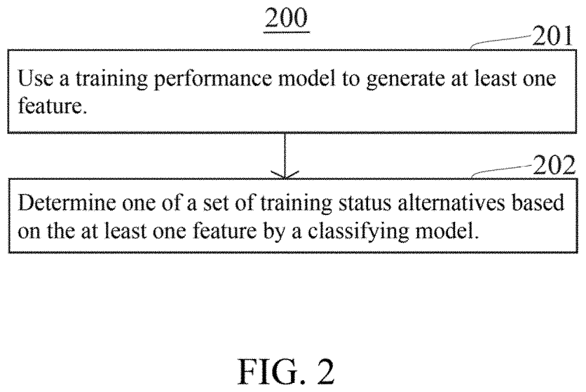

FIG. 2 illustrates a method 200 for determining a training status selected from a set of training status alternatives at a current time. Each of a set of training status alternatives has a corresponding physical condition. An overall training load in a duration before the current time is determined based on an exercise intensity measured by a sensing unit 101. The method 200 comprises:

Step 201: use a training performance model to generate at least one feature; and

Step 202: determine one of a set of training status alternatives based on the at least one feature by a classifying model.

Training Performance Model

The overall training load is determined in a duration before the current time based on an exercise intensity measured by a sensing unit 101. There are at least three terms (or parameters) in the training performance model. The first term represents a positive effect on the training performance (i.e. increasing training performance) at the current time determined based on the overall training load. The first term may be fitness, wellness, vigorousness or a combination thereof resulting from training (exercise training). For convenience of description, the first term may be represented in the form of the fitness resulting from training hereafter. The second term represents a negative effect on the training performance (i.e., decreasing training performance) at the current time determined based on the overall training load. The second term may be fatigue, tiredness, exhaust or a combination thereof resulting from training (exercise training). For convenience of description, the second term may be represented in the form of the fatigue resulting from training hereafter. The third term represents the training performance at the current time determined based on (a combination of) the first term and the second term. In other words, the third term represents a synthetic level resulting from all kinds of body responses (e.g., fitness or fatigue) to training (exercise training). In one embodiment, the third term representing the training performance may be determined based on a difference between the first term representing the positive effect on the training performance and the second term representing the negative effect on the training performance (e.g., subtract the second term from the first term). The third term represents the training performance may be determined based on a ratio of the first term representing the positive effect on the training performance to the second term representing the negative effect on the training performance. However, the present invention is not limited to the above cases; the first term and the second term can be disposed in any location of the training performance model such that the first term and the second term can respectively represent the positive effect and the negative effect on the training performance.

The training performance may have a reference training performance; a combination of the first term and the second term may be added to the reference training performance to generate the training performance. The following formula is an example of the training performance model.

P(t)=P(t₀ )+A_{<from t}_{₀} _{to t>}−B_{<from t}_{₀} _{to t>} (1)

P(t) is the training performance at the time point t (or at the current time), P(t₀) is the reference training performance at the time point t₀ before the time point t, A is the first term representing the positive effect on the training performance based on the overall training load in the duration T (T=t−t₀), and B is the second term representing the negative effect on the training performance based on the overall training load in the duration T (T=t−t₀).

The reference training performance may be divided into the reference first term and the reference second term. The following formula is an example of the training performance model.

P(t)=A(t₀)+A_{<from t}_{₀} _{to t>}−B(t₀)−B_{<from t}_{₀} _{to t>} (2)

P(t) is the training performance at the time point t (or at the current time), A(t₀) is the reference first term at the time point t₀ before the time point t, B(t₀) is the reference second term at the time point t₀, A is the first term representing the positive effect on the training performance based on the overall training load in the duration T (T=t−t₀), and B is the second term representing the negative effect on the training performance based on the overall training load in the duration T (T=t−t₀).

The first term representing the positive effect on the training performance may be determined based on the overall training load in a duration T and the second term representing the negative effect on the training performance may be determined based on the overall training load in a duration T′. The following formula is an example of the training performance model.

$$\begin{matrix}P ( t ) = A ( {t}_{0} ) + {A}_{< from {t}_{0} to t >} - B ( {t}_{0 ’} ) - {B}_{< from {t}_{0 ’} to t >} \\ = A ( {t}_{0} ) + {A}_{< from {t}_{0} to t >} - B ( {t}_{0 ’} ) - {B}_{< from {t}_{0 ’} to {t}_{0} >} - {B}_{from {t}_{0} t o t >} \\ = A ( {t}_{0} ) + {A}_{< from {t}_{0} to t >} - B ( {t}_{0} ) - {B}_{< from {t}_{0 ’} to t >}\end{matrix} \qquad ( 3 )$$

P(t) is the training performance at the time point t (or at the current time), A(t₀) is the reference first term at the time point t₀ before the time point t, B(t_{0′}) is the reference second term at the time point t_{0′} before the time point t, A in the top of the formula is the first term representing the positive effect on the training performance based on the overall training load in the duration T (T=t−t₀), B in the top of the formula is the second term representing the negative effect on the training performance based on the overall training load in the duration T′ (T′=t−t_{0′}). In this case, T′>T, the formula (3) can be regarded as the formula (2) after modifying the reference second term B(t_{0′}). In other words, if the duration of the overall training load of the first term is different from the duration of the overall training load of the second term, the overall training load of the first term and the overall training load of the second term can be adjusted to have the same duration after modifying the longer duration of the overall training load in one of the first term and the second term.

The first term may be determined based on a combination of the overall training load and a first overall training load setting (e.g., the positive-effect weighting factor and its component) and the second term may be determined based on a combination of the overall training load and a second overall training load setting (e.g., the negative-effect weighting factor and its component). The first overall training load setting may associate the over training load with the first term and the second overall training load setting may associate the over training load with the second term. At least one (or both) of the first overall training load setting of the first term and the second overall training load setting of the second term may be adjusted according to the training performance to precisely estimate the training performance. Precisely estimating the training performance further contributes to precisely estimating the training status. Each of the first overall training load setting and the second overall training load setting may and be represented in the form of the factor, the coefficient, the vector, the matrix or any other suitable setting. For convenience of description, each of the first overall training load setting and the second overall training load setting is represented in the form of the factor or the coefficient; however, the present invention is not limited to this case.

The duration of the overall training load may be divided into a plurality of time segments each of which comprises a training load (TL) therein. The sum of the training loads of the time segments is equal to the overall training load in the duration. Each time segment may have the same period or a different period. Preferably, each time segment may have the same period. The training load may be determined based on a plurality of exercise intensity zones. For example, each of the exercise intensity zones has a portion of the training load (e.g., the product of the exercise intensity and the exercise time) and the training load is a sum of the portions of the training load of the exercise intensity zones. In one embodiment, the training load may be represented in the form of a TRIMP (training impulse); however, the present invention is not limited to this case.

U.S. application Ser. No. 16/733,180 discloses that the exercise intensity zones are adjusted according to the fitness condition (e.g., VO₂ₘₐₓ or fitness performance level) of the user or adjusted based on the different fitness performance levels (see two-dimensional exercise intensity zones 100 and one-dimensional exercise intensity zones 101, 102 in U.S. application Ser. No. 16/733,180). Similar to U.S. application Ser. No. 16/733,180, the exercise intensity zones may be adjusted according to the training performance used as the third term of the training performance model. The training load may be modified based on the adjusted exercise intensity zones to precisely estimate the training performance. Precisely estimating the training performance further contributes to precisely estimating the training status.

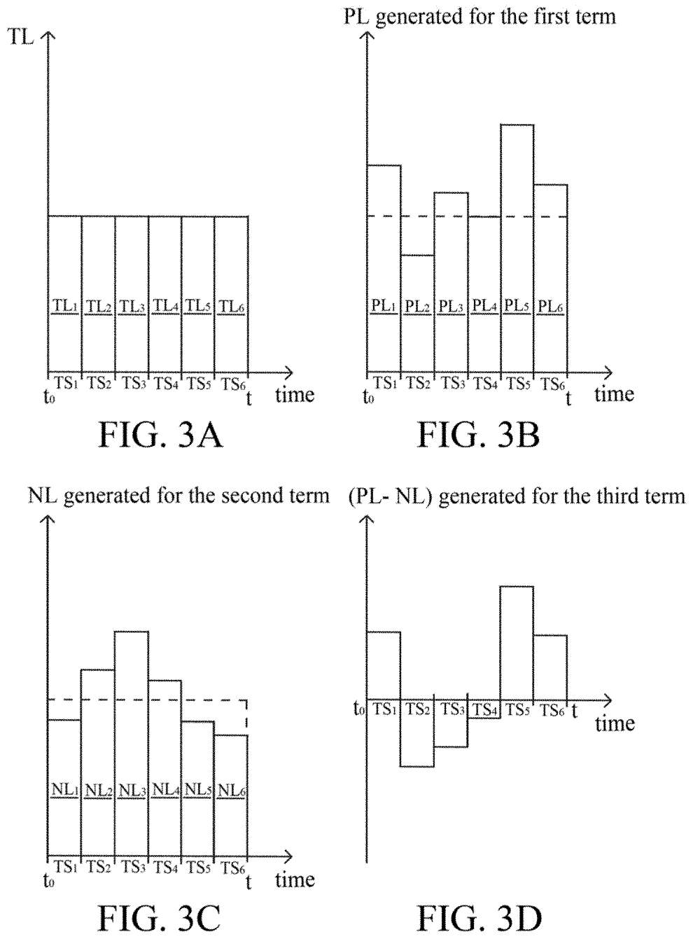

For each first time segment (each of TS₁, TS₂, TS₃, TS₄, TS₅ and TS₆) of the time segments, a positive-effect training load (the corresponding one of PL₁, PL₂, PL₃, PL₄, PL₅ and PL₆) is generated for the first term and a negative-effect training load (the corresponding one of NL₁, NL₂, NL₃, NL₄, NL₅ and NL₆) is generated for the second term (See FIGS. 3A to 3D, for example, TL₁=TL₂=TL₃=TL₄=TL₅=TL₆, the number of the time segments is 6). The positive-effect training load (each of PL₁, PL₂, PL₃, PL₄, P₅ and PL₆) is determined based on a combination of the training load (the corresponding one of TL₁, TL₂, TL₃, TL₄, TL₅ and TL₆) and a positive-effect weighting factor and the negative-effect training load (each of NL₁, NL₂, NL₃, NL₄, NL₅ and NL₆) is determined based on a combination of the training load (the corresponding one of TL₁, TL₂, TL₃, TL₄, TL₅ and TL₆) and a negative-effect weighting factor. In one embodiment, the positive-effect training load (each of PL₁, PL₂, PL₃, PL₄, PL₅ and PL₆) is the product of the training load (the corresponding one of TL₁, TL₂, TL₃, TL₄, TL₅ and TL₆) and the positive-effect weighting factor, and the negative-effect training load (each of NL₁, NL₂, NL₃, NL₄, NL₅ and NL₆) is the product of the training load (the corresponding one of TL₁, TL₂, TL₃, TL₄, TL₅ and TL₆) and the negative-effect weighting factor. The sum of the positive-effect training loads (PL₁, PL₂, PL₃, PL₄, PL₅ and PL₆) corresponding to the time segments (TS₁, TS₂, TS₃, TS₄, TS₅ and TS₆) is equal to the increasing training performance in the first term and the sum of the negative-effect training loads (NL₁, NL₂, NL₃, NL₄, NL₅ and NL₆) corresponding to the time segments (TS₁, TS₂, TS₃, TS₄, TS₅ and TS₆) is equal to the decreasing training performance in the second term. The following two formulas is an example of the first term and the second term of the training performance model.

A_{<from t}_{₀}_{to t>}=K_{A1}*TL₁+K_{A2}*TL₂+ . . . +K_{Ai}*TLᵢ+ . . . +K_{A(N-1)}*TL_{N-1}+K_{AN}*TL_{N} (4)

B_{<from t}_{₀}_{to t>}=K_{B1}*TL₁+K_{B2}*TL₂+ . . . +K_{Bj}+ . . . +K_{B(N-1)}*TL_{N-1}+K_{BN}*TL_{N} (5)

A is the first term representing the positive effect on the training performance based on the overall training load in the duration T (T=t−t₀), B is the second term representing the negative effect on the training performance based on the overall training load in the duration T (T=t−t₀), the duration T of the overall training load is divided into N time segments (time segment 1˜time segment N) each of which has a training load TLᵢ therein, the positive-effect training load K_{Ai}*TLᵢ is the product of the training load TLᵢ and the positive-effect weighting factor K_{Ai}, and the negative-effect training load K_{Bi}*TLᵢ is the product of the training load TLᵢ and the negative-effect weighting factor K_{Bi}.

The positive-effect weight factor may comprise a first positive-effect factor and the negative-effect weight factor may comprise a first negative-effect factor. The first positive-effect factor may use the time interval between the first time segment and the current time as the first influence time of the training load in the first time segment to represent the positive effect on the training performance and the first negative-effect factor may use the time interval between the first time segment and the current time as the first influence time of the training load in the first time segment to represent the negative effect on the training performance. Each of the first positive-effect factor and the first negative-effect factor may take into account a lapse of the time interval between the first time segment and the current time. Physiologically, when the time interval between the first time segment and the current time increases, the training load in the first time segment has a less effect on the training performance at the current time. Correspondingly, each of the first positive-effect factor and the first negative-effect factor decreases with the increase of the time interval between the first time segment and the current time. Each of the first positive-effect factor and the first negative-effect factor may be not more than 1 and not less than 0. The first positive-effect factor may be more than the first negative-effect factor because the fitness resulting from training decays more slowly than the fatigue resulting from training physiologically. In other words, for taking into account the lapse of time, the fitness resulting from training in the first time segment has a more effect on the training performance at the current time than the fatigue resulting from training. For example, the first positive-effect factor may be exp(−TIᵢ/τ_{A}) (τ_{A} is a time constant defined in the first term and TIᵢ is the time interval between the first time segment i and the current time) and the first negative-effect factor may be exp(−TIᵢ/τ_{B}) (τ_{B} is a time constant defined in the second term and TIᵢ is the time interval between the first time segment i and the current time); the time constant τ_{A} is more than the time constant τ_{B}.

Besides that the first positive-effect factor uses the time interval between the first time segment and the current time as the first influence time of the training load in the first time segment to represent the positive effect on the training performance and the first negative-effect factor uses the time interval between the first time segment and the current time as the first influence time of the training load in the first time segment to represent the negative effect on the training performance, there may be any other factor characterizing the positive-effect training load and the negative-effect training load. The positive-effect weight factor may further comprise a second positive-effect factor combined with the first positive-effect factor and the negative-effect weight factor may further comprise a second negative-effect factor combined with the first negative-effect factor; for example, the positive-effect weight factor may comprise a product of the first positive-effect factor and the second positive-effect factor and the negative-effect weight factor may comprise a product of the first negative-effect factor and the second negative-effect factor. The second positive-effect factor may (or may only) use the first time segment as the second influence time of the training load in the first time segment to represent the positive effect on the training performance and the second negative-effect factor may (or may only) use the first time segment as the second influence time of the training load in the first time segment to represent the negative effect on the training performance. The second positive-effect factor may be less than the second negative-effect factor because the fatigue resulting from training has a more effect on the training performance at the current time than the fitness resulting from training physiologically for taking into account using the first time segment as the second influence time of the training load in the first time segment to represent the positive/negative effect on the training performance. Physiologically, after the training load in the first time segment is calculated by an objective method (e.g., TRIMP (training impulse)), the actual physical load resulting from the fatigue may be different from the training load and the actual physical load resulting from the fitness may be different from the training load. Physiologically, the actual physical load resulting from the fatigue may be more than the actual physical load resulting from the fitness for taking into account using the first time segment as the second influence time of the training load in the first time segment to represent the positive/negative effect on the training performance, so the product of the second negative-effect factor and the training load may be more than the product of the second positive-effect factor and the training load in one embodiment. The following two formulas are an example of the first term and the second term of the training performance model. For convenience of description, the positive-effect weight factor may only comprise a product of the first positive-effect factor and the second positive-effect factor and the negative-effect weight factor only comprises a product of the first negative-effect factor and the second negative-effect factor; however, the present invention is not limited to this case, for example the positive-effect weight factor may comprise a successive product of the first positive-effect factor, the second positive-effect factor and the third positive-effect factor; the negative-effect weight factor may comprise a successive product of the first negative-effect factor, the second negative-effect factor and the third negative-effect factor.

$${A}_{< from {t}_{0} to t >} = {K}_{A 11} * {K}_{A 12} * {TL}_{1} + {K}_{A 21} * {K}_{A 22} * {TL}_{2} + … + {K}_{Ai 1} * {K}_{Ai 2} * {TL}_{i} + … + {K}_{A ( N - 1 ) 1} * {K}_{A ( N - 1 ) 2} * {TL}_{N - 1} + {K}_{AN 1} * {K}_{AN 2} * {TL}_{N} \qquad ( 6 )$$

$$if {K}_{A 11} = {K}_{A 21} = … = {K}_{Ai 1} = … = {K}_{A ( N - 1 ) 1} = {K}_{AN 1} = {K}_{A 1} ,$$

$${A}_{< from {t}_{0} to t >} = {K}_{A 1} ( {K}_{A 12} * {TL}_{1} + {K}_{A 22} * {TL}_{2} + … + {K}_{Ai 2} * {TL}_{i} + … + {K}_{A ( N - 1 ) 2} * {TL}_{N - 1} + {K}_{AN 2} * {TL}_{N} ) \qquad ( 7 )$$

$${B}_{< from {t}_{0} to t >} = {K}_{B 11} * {K}_{B 12} * {TL}_{1} + {K}_{B 21} * {K}_{B 22} * {TL}_{2} + … + {K}_{Bi 1} * {K}_{Bi 2} * {TL}_{i} + … + {K}_{B ( N - 1 ) 1} * {K}_{B ( N - 1 ) 2} * {TL}_{N - 1} + {K}_{BN 1} * {K}_{BN 2} * {TL}_{N} \qquad ( 8 )$$

$$if {K}_{B 11} = {K}_{B 21} = … = {K}_{Bi 1} = … = {K}_{B ( N - 1 ) 1} = {K}_{BN 1} = {K}_{B 1} ,$$

$${B}_{< from {t}_{0} to t >} = {K}_{B 1} ( {K}_{B12} * {TL}_{1} + {K}_{B 22} * {TL}_{2} + … + {K}_{Bi 2} * {TL}_{i} + … + {K}_{B ( N - 1 ) 2} * {TL}_{N - 1} + {K}_{BN 2} * {TL}_{N} ) \qquad ( 9 )$$

A is the first term representing the positive effect on the training performance based on the overall training load in the duration T (T=t−t₀), B is the second term representing the negative effect on the training performance based on the overall training load in the duration T (T=t−t₀), the duration T of the overall training load may be divided into N time segments (time segment 1˜time segment N) each of which has a training load TLᵢ therein, K_{Ai1}*K_{Ai2} is the positive-effect weighting factor associating the training load TLᵢ in the time segment i with the first term, K_{Ai1} is the first positive-effect factor associating the training load TLᵢ in the time segment i with the first term, K_{Ai2} is the second positive-effect factor associating the training load TLᵢ in the time segment i with the first term, K_{Bi1}*K_{Bi2} is the negative-effect weighting factor associating the training load TLᵢ in the time segment i with the second term, K_{Bi1} is the first negative-effect factor associating the training load TL₁ in the time segment i with the second term, and K_{Bi2} is the second negative-effect factor associating the training load TLᵢ in the time segment i with the second term.

In the one embodiment of the present invention, the positive-effect weight factor may further comprise a third positive-effect factor (combined with the first positive-effect factor) and the negative-effect weight factor may further comprise a third negative-effect factor (combined with the first negative-effect factor); for example, the positive-effect weight factor may comprise a product of the first positive-effect factor and the third positive-effect factor and the negative-effect weight factor may comprise a product of the first negative-effect factor and the third negative-effect factor. In one embodiment of the present invention, the positive-effect weight factor may further comprise a third positive-effect factor combined with the first positive-effect factor and the second positive-effect factor and the negative-effect weight factor may further comprise a third negative-effect factor combined with the first negative-effect factor and the second negative-effect factor; for example, the positive-effect weight factor may comprise a successive product of the first positive-effect factor, the second positive-effect factor and the third positive-effect factor and the negative-effect weight factor may comprise a successive product of the first negative-effect factor, the second negative-effect factor and the third negative-effect factor. At least one second time segment (of the time segments) is between the first time segment and the current time; the second positive-effect factor may take into account the training load in each of the at least one second time segment being not less than a threshold H₁ or not more than a threshold H₂ and the second negative-effect factor may take into account the training load in each of the at least one second time segment being not less than a threshold H₃ or not more than a threshold H₄; the threshold H₁ is more than the threshold H₂ and the threshold H₃ is more than the threshold H₄.

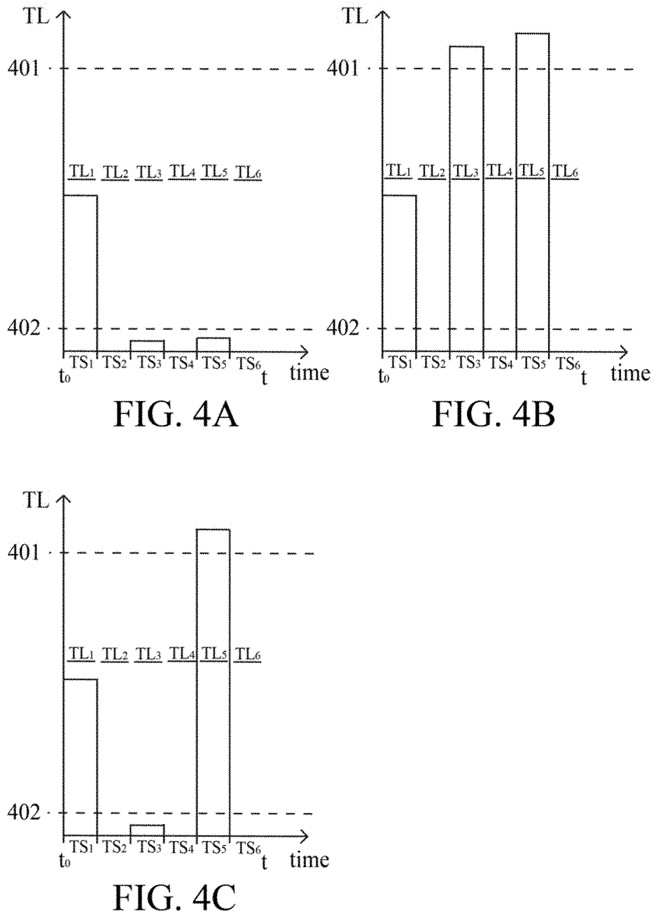

FIGS. 4A to 4C illustrates some cases in which each of the third positive-effect factor and the third negative-effect factor needs taking into account. For convenience of description, take the third positive-effect factor for example; the same reason can be applied in taking the third negative-effect factor for example. The first positive-effect factor only takes into account the length of the time interval (e.g., TS₂˜TS₆) between the first time segment (e.g., TS₁) and the current time t but doesn't take into account at least one training load (e.g., TL₂˜TL₆) distributed in the time interval (e.g., TS₂˜TS₆) between the first time segment (e.g., TS₁) and the current time t. If at least one training load (e.g., TL₂˜TL₆) in the time interval (e.g., TS₂˜TS₆) between the first time segment (e.g., TS₁) and the current time t have extreme value(s) which is low or high enough to have a further obvious effect on the training performance, it is taken into account by defining the third positive-effect factor to precisely estimate the training performance. Precisely estimating the training performance further contributes to precisely estimating the training status. In one embodiment, when the training load is not less than a threshold 401, it is high enough to have a further obvious effect on the training performance; when the training load is not more than a threshold 402 (e.g., the threshold 402 may be 0), it is low enough to have a further obvious effect on the training performance. FIG. 4A illustrates that the training loads TL₂˜TL₆ are low; FIG. 4B illustrates that the training loads TL₂, TL₄, TL₆ are low and TL₃, TL₅ are high; FIG. 4C illustrates that the training loads TL₂, TL₃, TL₄, TL₆ are low and TL₅ is high. The threshold 401 and the threshold 402 may be adjusted according to the training performance.

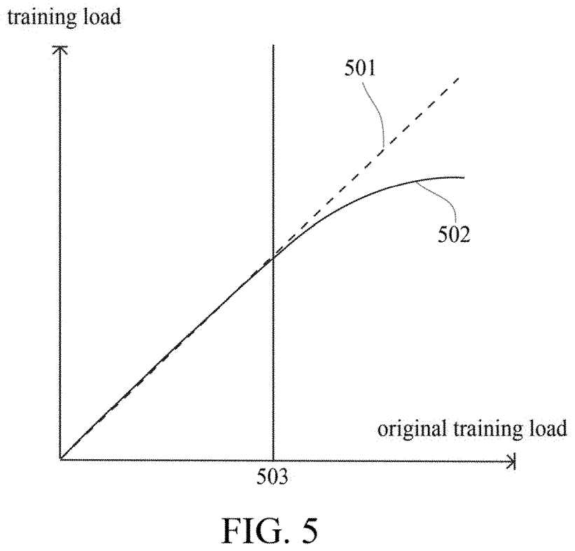

In the one embodiment of the present invention, each of the time segments may further comprise an original training load therein, wherein the sum of the original training loads of the time segments is equal to the overall training load in the duration; because the body has a finite training capacity physiologically (i.e., the training load in the time segment has a maximum) when the person does his best to train himself, the original training load (e.g., by an objective method e.g., TRIMP (training impulse)) is modified to be the training load (e.g., according to the finite training capacity of the body) if the original training load is more than a threshold 503 (see the solid lone 502 in FIG. 5, the slope of the dashed line 501 is 1). The threshold 503 may be adjusted according to the training performance because the finite training capacity of the body varies with the training performance.

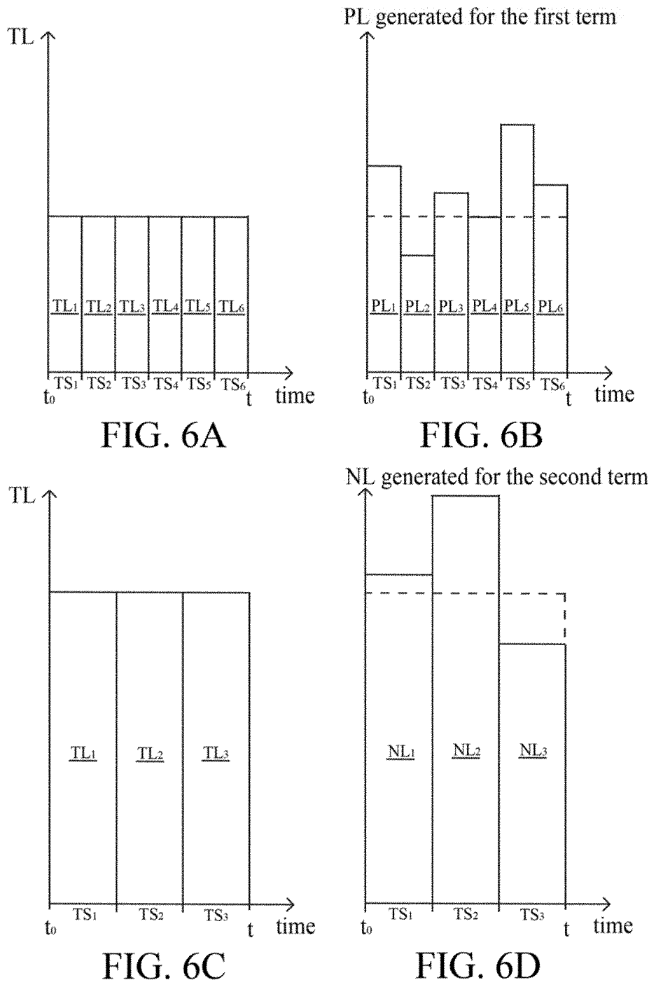

In another embodiment of the present invention, the positive-effect training load may be generated for the first term for each time segment of N time segments into which the duration of the overall training load is divided and the negative-effect training load may be generated for the second term for each time segment of M time segments into which the duration of the overall training load is divided (N is not equal to M) (see FIGS. 6A to 6D, N=6 and M=3 in this case). The training performance model with the different time segment number in the first term and the second term is similarly to the training performance model with the same time segment number in the first term and the second term, so it doesn't be described in detailed therein.

Classifying Model

After building up the training performance model, generate at least one feature based on at least one of the first term, the second term and the third term (in step 202). At least one feature may be generated based on the first term, the second term and the third term. At least one feature may comprise at least one of a first variance of the first term, a second variance of the second term and a third variance of the third term. At least one feature may comprise a first variance of the first term, a second variance of the second term and a third variance of the third term. The variance may be represented in the form of a trend or a standard deviation. At least one feature may comprise any other statistical data different from the variance of at least one of the first term, the second term and the third term. For example, the statistical data can be a mean or a relative ratio.

After generating at least one feature based on at least one of the first term, the second term and the third term (in step 201), determining one of a set of training status alternatives based on the at least one feature by a classifying model (in step 202). The training status alternatives may comprise detraining, unproductive, overreaching, maintaining, recovery, peaking, productive or any other suitable alternatives. The classifying model describes that a set of training status alternatives are associated with the at least one features. The classifying model may be built up by a machine-learning method including decision tree, support vector machine, linear regression, logistic regression or neural network. The classifying model may be built up by any other suitable method. For example, the training status alternatives comprising detraining, unproductive, overreaching, maintaining, recovery, peaking and productive are associated with at least one of a first variance (trend) of the first term, a second variance (trend) of the second term and a third variance (trend) of the third term in the following way; however, the present invention is not limited to this case.

Detraining

A loss of training-induced positive physiological adaptations (i.e., VO_{2max,} aerobic endurance, running economy etc.) due to a constant degree of training reduction or cessation.

The training status is detraining if the following limitations are met in a duration: the first term decreases by more than a threshold A₁₁; the second term decreases by more than a threshold A₁₂; and the third term decreases by more than a threshold A₁₃ (Each of the thresholds A₁₁, A₁₂, A₁₃ is a positive number).

Unproductive

Current training load negated the previous improvements on physiological adaptations.

The training status is unproductive if the following limitations are met in a duration: the first term increases by less than a threshold A₂₁₁ and decreases by less than a threshold A₂₁₂; the second term increases by more than a threshold A₂₂; and the third term increases by less than a threshold A₂₃₁ and decreases by less than a threshold A₂₃₂

(Each of the thresholds A₂₁₁, A₂₁₂, A₂₂, A₂₃₁, A₂₃₂ is a positive number).

Overreaching

A short-term physiological status with a series of high training load resulting in negative training adaptations.

The training status is overreaching if the following limitation is met in a duration: the second term increases by more than a threshold A₃₂ (The thresholds A₃₂ is a positive number).

Maintaining

Current training load is sufficient to maintain previous improvements on physiological adaptations.

The training status is maintaining if the following limitations are met in a duration: the first term increases by less than a threshold A₄₁₁ and decreases by less than a threshold A₄₁₂; the second term increases by less than a threshold A₄₂₁ and decreases by less than a threshold A₄₂₂; and the third term increases by less than a threshold A₄₃₁ and decreases by less than a threshold A₄₃₂ (Each of the thresholds A₄₁₁, A₄₁₂, A₄₂₁, A₄₂₂, A₄₃₁, A₄₃₂ is a positive number).

Recovery

A status of physiological restorative process with reduction/cessation of training stress/load.

The training status is recovery if the following limitations are met in a duration: the first term increases by less than a threshold A₅₁₁ and decreases by less than a threshold A₅₁₂; the second term decreases by more than a threshold A₅₂; and the third term increases by more than a threshold A₅₃. (Each of the thresholds A₅₁₁, A₅₁₂, A₅₂, A₅₃ is a positive number).

Peaking

A status of optimal sports performance induced from a reduction of training load and concomitant attenuation of physiological fatigue/stress.

The training status is peaking if the following limitations are met in a duration: the first term decreases by more than a threshold A₆₁; the second term decreases by more than a threshold A₆₂; and the third term increases by more than a threshold A₆₃ (Each of the thresholds A₆₁, A₆₂, A₆₃ is a positive number).

Productive

Current training load induced beneficial effects on physiological conditions (e.g., performance or fitness).

The training status is productive if the following limitations are met in a duration: the first term increases by more than a threshold A₇₁; the second term increases by more than a threshold A₇₂; and the third term increases by more than a threshold A₇₃ (Each of the thresholds A₇₁, A₇₂, A₇₃ is a positive number).

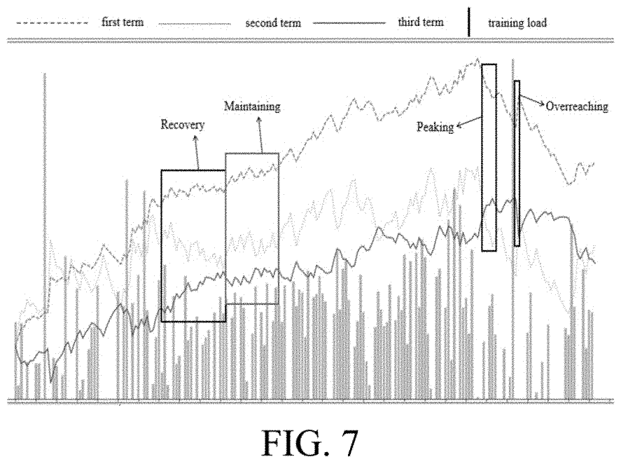

FIG. 7 illustrates some of the training status alternatives and the corresponding the limitations associated with a first trend of the first term, a second trend of the second term and a third trend of the third term.

The above disclosure is related to the detailed technical contents and inventive features thereof. People skilled in the art may proceed with a variety of modifications and replacements based on the disclosures and suggestions of the invention as described without departing from the characteristics thereof. Nevertheless, although such modifications and replacements are not fully disclosed in the above descriptions, they have substantially been covered in the following claims as appended.

1. A method for determining a training status selected from a set of training status alternatives each of which has a corresponding physical condition at a current time, the method comprising:
determining an overall training load in a duration before the current time based on an exercise intensity measured by a sensing unit;
determining, by a processing unit, a first term representing a positive effect on a training performance based on the overall training load and a second term representing a negative effect on the training performance based on the overall training load;
determining, by the processing unit, a third term representing the training performance based on the first term and the second term;
generating, by the processing unit, at least one feature based on at least one of the first term, the second term and the third term; and
determining, by the processing unit, one of the set of training status alternatives based on the at least one feature by a classifying model describing that the set of training status alternatives are associated with the at least one features.

2. The method according to claim 1, wherein the first term is determined based on a combination of the overall training load and a first overall training load setting and the second term is determined based on a combination of the overall training load and a second overall training load setting, wherein the first overall training load setting associates the over training load with the first term and the second overall training load setting associates the over training load with the second term.

3. The method according to claim 1, wherein at least one of the first overall training load setting of the first term and the second overall training load setting of the second term is adjusted according to the training performance.

4. The method according to claim 1, wherein the duration is divided into a plurality of time segments each of which comprises a training load therein, wherein for each first time segment of the plurality of time segments, a positive-effect training load is generated for the first term and a negative-effect training load is generated for the second term, wherein the positive-effect training load is determined based on a combination of the training load and a positive-effect weighting factor and the negative-effect training load is determined based on a combination of the training load and a negative-effect weighting factor.

5. The method according to claim 4, wherein a sum of the training loads of the time segments is equal to the overall training load in the duration.

6. The method according to claim 4, wherein the positive-effect weight factor comprises a first positive-effect factor and the negative-effect weight factor comprises a first negative-effect factor, wherein the first positive-effect factor uses a time interval between the first time segment and the current time as an first influence time of the training load in the first time segment to represent the positive effect on the training performance and the first negative-effect factor uses the time interval between the first time segment and the current time as the first influence time of the training load in the first time segment to represent the negative effect on the training performance.

7. The method according to claim 4, wherein the positive-effect weight factor comprises a first positive-effect factor and the negative-effect weight factor comprises a first negative-effect factor, wherein each of the first positive-effect factor and the first negative-effect factor takes into account a lapse of a time interval between the first time segment and the current time.

8. The method according to claim 7, wherein each of the first positive-effect factor and the first negative-effect factor decreases with an increase of the time interval between the first time segment between the current time.

9. The method according to claim 7, wherein the first positive-effect factor is more than the first negative-effect factor.

10. The method according to claim 6, wherein the positive-effect weight factor further comprises a second positive-effect factor combined with the first positive-effect factor and the negative-effect weight factor further comprises a second negative-effect factor combined with the first negative-effect factor, wherein the second positive-effect factor uses the first time segment as an second influence time of the training load in the first time segment to represent the positive effect on the training performance and the second negative-effect factor uses the first time segment as the second influence time of the training load in the first time segment to represent the negative effect on the training performance.

11. The method according to claim 10, wherein the positive-effect weight factor comprises a first product of a first positive-effect factor and a second positive-effect factor and the negative-effect weight factor comprises a second product of a first negative-effect factor and a second negative-effect factor.

12. The method according to claim 10, wherein the second positive-effect factor is less than the second negative-effect factor.

13. The method according to claim 6, wherein the positive-effect weight factor further comprises a second positive-effect factor combined with the first positive-effect factor and the negative-effect weight factor further comprises a second negative-effect factor combined with the first negative-effect factor, wherein at least one second time segment is between the first time segment and the current time, wherein the second positive-effect factor takes into account the training load in each of the at least one second time segment being not less than a first threshold or not more than a second threshold and the second negative-effect factor takes into account the training load in each of the at least one second time segment being not less than a third threshold or not more than a fourth threshold, wherein the first threshold is more than the second threshold and the third threshold is more than the fourth threshold.

14. The method according to claim 4, wherein each of the plurality of time segments further comprises an original training load therein, wherein a sum of the original training loads of the time segments is equal to the overall training load in the duration, wherein if an original training load is more than a first threshold, the original training load is modified to be the training load.

15. The method according to claim 14, wherein if the original training load is more than a first threshold, the original training load is modified to be the training load according to a finite training capacity of a body.

16. The method according to claim 1, wherein the at least one feature comprises at least one of a first variance of the first term, a second variance of the second term and a third variance of the third term.

17. The method according to claim 1, wherein the classifying model is built up by a machine-learning method.

18. The method according to claim 1, wherein the duration is divided into a plurality of time segments each of which comprises a training load therein, wherein the training load is determined based on a plurality of exercise intensity zones, wherein the plurality of exercise intensity zones are adjusted according to the training performance.

19. The method according to claim 4, wherein a first sum of the positive-effect training loads corresponding to the plurality of time segments is equal to an increasing training performance in the first term and a second sum of the negative-effect training loads corresponding to the plurality of time segments is equal to a decreasing training performance in the second term.

20. A method for determining a training performance at a current time, the method comprising:
determining an overall training load in a duration before the current time based on an exercise intensity measured by a sensing unit;
determining, by a processing unit, a first term representing a positive effect on the training performance based on the overall training load and a second term representing a negative effect on the training performance based on the overall training load; and
determining, by the processing unit, a third term representing the training performance based on the first term and the second term;
wherein the duration is divided into a plurality of time segments each of which comprises a training load therein, wherein for each first time segment of the plurality of time segments, a positive-effect training load is generated for the first term and a negative-effect training load is generated for the second term, wherein the positive-effect training load is determined based on a combination of the training load and a positive-effect weighting factor and the negative-effect training load is determined based on a combination of the training load and a negative-effect weighting factor;
wherein the positive-effect weight factor comprises a first positive-effect factor decreasing with an increase of a time interval between the first time segment between the current time and the negative-effect weight factor comprises a first negative-effect factor decreasing with the increase of the time interval between the first time segment between the current time;
wherein the positive-effect weight factor further comprises a second positive-effect factor combined with the first positive-effect factor and the negative-effect weight factor further comprises a second negative-effect factor combined with the first negative-effect factor, wherein at least one second time segment is between the first time segment and the current time, wherein the second positive-effect factor takes into account the training load in each of the at least one second time segment being not less than a first threshold or not more than a second threshold and the second negative-effect factor takes into account the training load in each of the at least one second time segment being not less than a third threshold or not more than a fourth threshold, wherein the first threshold is more than the second threshold and the third threshold is more than the fourth threshold.
